(19) United States
(12) Patent Application Publication
(10) Pub. No.: US 2025/0255842 A1
(43) Pub. Date: Aug. 14, 2025

(54) SPECIALIZED PRO-RESOLVING LIPID MEDIATORS FOR TREATING LONG COVID-19

(71) Applicant: Solutex GC, S.L., Madrid (ES)

(72) Inventors: Pedro Antonio Regidor, Madrid (ES); José Miguel Rizo, Madrid (ES); Rocío Gutierrez, Madrid (ES)

(21) Appl. No.: 18/850,472

(22) PCT Filed: Mar. 24, 2023
(86) PCT No.: PCT/EP2023/057737

§ 371 Date: Sep. 24, 2024

(30) Foreign Application Priority Data
Mar. 25, 2022 (EP) 22382280.0

(51) Int. Cl. A61K 31/202; A61P 31/14

(52) U.S. Cl. CPC A61K 31/202; A61P 31/14

(57) ABSTRACT

The present invention concerns a treatment of Long COVID-19 by using specialized pro-resolving lipid mediators (SPM) or their active precursors. optionally in combination with a non-steroidal anti-inflammatory drug.

### CROSS REFERENCE TO RELATED APPLICATIONS

This application is a U.S. National Phase Application of PCT International Application Number PCT/EP2023/057737, filed on Mar. 24, 2023, designating the United States of America and published in the English Language, which is an International Application of and claims the benefit of priority to European Patent Application No. 22382280.0, filed on Mar. 25, 2022. The disclosures of the above-referenced applications are hereby expressly incorporated by reference in their entireties.

### FIELD OF THE INVENTION

The present invention concerns the use of specialized pro-resolving lipid mediators or their active precursors in the treatment of Long COVID-19 and/or inflammation associated with Long COVID-19.

### BACKGROUND OF THE INVENTION

In many chronic diseases, including vascular and neurological disorders, as well as metabolic syndrome, excessive inflammatory processes are manifested, thus representing a public health concern. When a host experiences a trauma, barrier breakage, or microbial invasion, potential invaders must be eliminated, the location must be cleared, and affected tissue must be remodelled and regenerated. For the acute inflammatory response, several lipid mediators are crucial. They include eicosanoids (prostaglandins and leukotrienes), which derive from arachidonic acid (ARA), an essential fatty acid [1,2], and different cytokines and chemokines [3-5]. These molecules interact with each other, thereby further intensifying the inflammatory process that may, in turn, be counteracted with pharmacological inhibitors and receptor antagonists.

Historically, the inflammatory response used to be separated into an active initiation and a passive resolution process [6]. Recently, however, mediators were identified which have pro-resolving capacities and can be synthesized from omega-3 (n-3) essential fatty acids (EFA). Studies have shown that the resolution process can be “switched on” in animal models and may thus rather be an active response in the self-limitation of acute inflammation than a passive dilution of chemo-attractants [7, 8].

Molecules, which are supposed to act as mediators, must be supplied in sufficient amounts in order to lead to reactions in vivo. For eicosapentaenoic acid (EPA) and docosahexaenoic acid (DHA), anti-inflammatory properties have been proposed for many years. These omega-3 fatty acids compete with arachidonic acid in reducing pro-inflammatory eicosanoids [9]. However, the underlying molecular mechanisms had remained obscure until recent results emerged, and whether EPA or DHA is more relevant for human health or therapeutic options is still under debate [9].

It has been shown for resolving inflammatory exudates that omega-3 fatty acids serve as substrates for the synthesis of specific signalling molecules—the so-called specialized pro-resolving mediators (SPMs), which comprise resolvins, protectins, lipoxins, and maresins [10]. These findings triggered new studies concerning the resolution pathways and the immune mechanisms underlying homeostasis. It was shown in animal models that SPMs promote critical paths of the inflammatory resolution, as they limit the infiltration of polymorphonuclear neutrophils and the elimination of apoptotic cells by macrophages [11].

Inflammations may be resolved entirely or become a chronic state. Previously, resolution of active inflammation has been considered a passive event, upon which inflammatory mediators such as prostaglandins or cytokines were merely diluted, thus disappearing from the site of inflammation. This would finally lead to prevent leukocyte infiltration into the tissue. However, Serhan et al. provided new evidence to revise this theory by demonstrating the existence of an active resolution process mediated by so-called selective pro-resolving mediators (SPMs) in several studies. The SPM molecular superfamily contains subgroups named resolvins (Rv), protectins (PD), maresins (MaR), and lipoxins (LX). SPMs are crucial for sufficient resolution of inflammatory processes, and based on these new findings, Serhan et al. described in detail novel pathways for the potential development of acute inflammation. They include the action of the SPMs [12].

Importantly, within this new perception of inflammatory processes, the resolution is an active mechanism, which does not start with a delay, but at experimental timepoint Zero.

Alfa signals Omega throughout the course of inflammation, mainly SPMs were found to repress inflammatory signals by ending tissue infiltration of neutrophils and preventing further recruitment of immune cells to the site of inflammation. Subsequently, phagocytic macrophages are stimulated, which further leads to increased clearance and elimination of apoptotic polymorphonuclear neutrophils (PMNs) by efferocytosis and phagocytosis [4]

SPMs are biosynthesized from eicosapentaenoic acid (EPA, C20:5n-3) and docosahexaenoic acid (DHA, C22:6n-3). Both are omega-three polyunsaturated fatty acids (PUFAs) and serve as precursors in the biochemical pathways leading to SPMs via the metabolites 18-hydroxyeicosapentaenoic acid (18-HEPE), 17-hydroxydocosahexaenoic acid (17-HDHA), or 14-hydroxydocosahexaenoic acid (14-HDHA)[13].

SPMs or their active precursors, 18-HEPE, 17-HDHA and 14-HDHA, could therefore potentially be used to elicit dynamic resolution of inflammation. In contrast to traditionally applied anti-inflammatory therapies, they do not act as immunosuppressors, and debris is cleared, thus being potentially useful for the treatment of chronic inflammation. Substances, which are currently applied, have distinct disadvantages: steroids may interfere with wound healing, can promote osteoporosis, and is immunosuppressive. Non-steroidal anti-inflammatory drugs (NSAIDs) may lead to stomach bleeding, are potentially toxic for the cardiovascular system and the kidneys and interfere with wound healing. Cyclooxygenase 2 (COX-2) inhibitors constitute a risk factor for cardiovascular and thromboembolic events. Anti-TNF therapies for blocking cytokines lead to increased rates of infections and enhance the risk of lymphoma development.

SPMs, on the other hand, were shown to increase the killing of microbial invaders and their clearance by immunocytes. It was demonstrated that they down-regulate infiltration and recruitment of PMN, enhance phagocytosis, and efferocytosis (M1 to M2). Application of SPMs also decreased the level of pro-inflammatory chemical mediators, while increasing the number of anti-inflammatory mediators like IL-10, for example. Finally, they can reduce inflammatory pain, stimulate the regeneration of inflamed tissue, and promote wound healing [12].

COVID-19 is a new disease caused by coronavirus SARS-COV-2. It was first described in 2019, developed into an epidemic in January 2020 and has spread the globe to the current COVID-19 pandemic. The disease histories are non-specific and vary greatly. In addition to symptomless infections, mainly mild to moderate histories were observed, but also severe ones with pneumonia on both sides, including lung failure, multiorgan failure and death. Even mild disease histories can lead to long-term damage. Thus far, there are no specific therapeutic agents for coronavirus infections [14].

Currently, there is no specific treatment for COVID-19, so healthcare providers treat the clinical symptoms (e.g. fever, difficulty breathing) of patients. Supportive care (e.g. fluid management, oxygen therapy, etc.) can be highly effective for patients with symptoms. Specifically, there are currently no antiviral drugs recommended or licensed by WHO or any government regulatory department for COVID-19 as such. Clinical studies of some drugs (human interferon alpha-2b, ribavirin, chloroquine phosphate, lopinavir and arbidol) have undergone testing of the efficacy and safety of these drugs in the treatment of COVID-19 [15].

Enhanced plasma concentrations of proinflammatory cytokines have been found in COVID-19 patients with need for intensive care compared to non-ICU patients in early reports of COVID-19 patients [14]. These inflammatory conditions may lead to subsequent activation of the coagulation response as measured by increased levels of D-Dimers, a characteristic parameter for pro-coagulatory conditions. Elevated concentrations of D-Dimers have been linked to increased mortality of COVID-19 patients and both septic patients as well as those developing DIC conditions are at a higher risk for a fatal course of the disease. Although the mechanisms leading to coagulation during SARS-COV-2 infection have not been elucidated yet, they rather seem to relate to the inflammatory response of the host instead of distinct viral pathogenic factors and in contrast to RNA-stranded viruses associated with haemorrhagic fever like Ebola, infections with SARS-CoV-2 do not result in excessive bleeding [14]. Data from Wuhan support the view that Covid-19-related coagulopathy rather results from the inflammatory host response that leads to exaggerated thrombotic processes through the above-described thrombo-inflammatory interrelations of different host signalling pathways.

Recent data of Nicolai et al. present Covid-19 as a disease with a dysregulated immunothrombosis driven by activated neutrophils and immunogenic platelets that contribute to organ injury and a systemic thrombogenic state in COVID-19. In addition, they found that plasmatic coagulation is skewed towards a procoagulant state correlating with disease severity, reflected by peripheral blood coagulation tests as well as histopathological evidence of microvascular thrombosis in affected organs. They stated that platelets, neutrophils, and the coagulation cascade are drivers of disease severity and might prove to be valuable pharmacological targets in COVID-19. In addition, SARS-COV-2 infected patients are at risk for increased thrombotic events, making prophylactic anticoagulation and vigilant monitoring for thrombotic complications a central task in management of COVID-19 patients.

Cherpocova et al. could show that the administration of resolvin D4 (RvD4), an SPM that was enriched at the natural onset of thrombus resolution, significantly reduced thrombus burden, with significantly less neutrophil infiltration and more pro-resolving monocytes in the thrombus, as well as an increased number of cells in an early apoptosis state. Moreover, RvD4 promoted the biosynthesis of other D-series resolvins involved in facilitating resolution of inflammation, and they suggest that delivery of SPMs, specifically RvD4, modulates the severity of thromboinflammatory disease in vivo and improves thrombus resolution [17].

Serhan et al. also could show under a long-term treatment the safety of the SPMs. This trial was intended to improve de-clotting in coronary arterial disease and no severe adverse events were reported. Therefore, SPMs can be considered as safe [18].

COVID-19 symptoms, such as fatigue, dyspnea, asthenia, joint pain, and chest pain, may persist long after the virus has been cleared from the body of patients. When symptoms persist more than 12 weeks after the onset of symptoms, it is referred to as “Long COVID-19”, “chronic COVID-19” or “post-COVID-19 syndrome”. Long COVID-19 is a serious impediment for COVID-19 patients to return to a normal life. Accordingly, it is an objective of the present invention to provide treatment and/or resolution of Long COVID-19.

Without being bound by a particular theory, the present inventors have found that Long COVID-19 is characterized by depleted levels of SPMs and a low ratio of SPMs to proinflammatory cytokines and that therefore administration of 17-HDHA, 14-HDHA, 18-HEPE, or SPMs per se could be effective in restoring SPM levels in Long COVID-19 patients.

### SUMMARY OF THE INVENTION

In one aspect, the present invention concerns a composition for use in the treatment of Long COVID-19, wherein said composition comprises a specialized pro-resolving lipid mediator (SPM), an SPM precursor and/or a pharmaceutically acceptable salt thereof, as well as any stereoisomer thereof.

In another aspect, the present invention concerns a method of treating Long COVID-19 by administration of a therapeutically effective amount of a composition comprising one or more of a specialized pro-resolving lipid mediator (SPM), an SPM precursor and/or a pharmaceutically acceptable salt thereof, as well as any stereoisomer thereof.

The treatment of Long COVID-19 may further comprise the administration of a non-steroidal anti-inflammatory drug (NSAID), such as acetylsalicylic acid. Without being bound by a particular theory, the non-steroidal anti-inflammatory drug, such as acetyl salicylic acid, functions as a co-enzyme for lipoxygenase and thus contributes to the biosynthesis of SPMs.

### BRIEF DESCRIPTION OF THE FIGURES

FIG. 1: Evolution of 17-HDHA and 18-HEPE concerntrations in serum. *P<0.05.

FIG. 2: Percentage of change: Monohydroxylated SPM precursors vs pro-inflammatory mediators.

FIG. 3: Composite metabolomes vs prostaglandins.

### DETAILED DESCRIPTION OF THE INVENTION

### Definitions

In the present context, the term “SPM” or “specialized pro-resolving lipid mediator” is intended to mean a compound which is a resolvin, a protectin, a lipoxin, or a maresin.

In the context of the present invention, the term “resolvin” is intended to cover resolvins resulting from the cascade of EPA, also referred to as “E series resolvins”, and from the cascade of DHA, also referred to as “D series resolvins”. Examples of E series resolvins include the compounds RvE1, RvE2, and RvE3. Examples of D series resolvins include the compounds RvD1, RvD2, RvD3, RvD4, RvD5, and RvD6.

In the context of the present invention, the term “protectin” is intended to cover protectins resulting from the cascade of DHA. Protectins include the compounds PD1, and PDX.

In the context of the present invention, the term “maresin” is intended to cover maresins resulting from the cascade of DHA. Maresins include the compounds MaR1, MaR2, MaR3, MCTR1, MCTR2, and MCTR3.

In the context of the present invention, the term “SPM precursor” or “SPM precursors” refers to 18-hydroxyeicosapentaenoic acid (18-HEPE), 17-hydroxydocosahexaenoic acid (17-HDHA), and/or 14-hydroxydocosahexaenoic acid (14-HDHA).

In the context of the present invention, the term “purified fish oil” is to be understood as an oil rich in omega-3 fatty acids deriving from marine sources, such as fish, crustacea (krill), mollusks, (squid), etc. The well-known textbook Bailey's Industrial Oil and Fat Products, Volumes 1-6, Set, 6th Edition, 2005 discloses methods of preparing purified fish oil.

In the context of the present invention, the term “purified algae oil” or “purified microalgae oil” is to be understood as an oil rich in omega-3 fatty acids deriving from algae sources or microalgae such as Thraustochytrids, Crypthecodinium cohnii, Ulkenia sp, Schizochytrium sp, etc.

In the context of the present invention, the term “purified vegetable oil” is to be understood as an oil rich in omega-3 fatty acids deriving from fruits, seeds, grains, and nuts, such as canola, coconut, corn, cottonseed, olive, palm, palm-kernel, peanut, safflower, soybean, sunflower, etc.

The compounds of the present invention can be in a free form or in the form of a pharmaceutically acceptable salt. In the context of the present invention, the term “pharmaceutically acceptable salt” is to be understood as a salt formed with the acid group of the poly-unsaturated fatty acids (PUFAs) comprised by the SPMs and SPM precursors, wherein the resulting counter-ion does not significantly add to the toxicity of the compound of the present invention. Examples of pharmaceutically acceptable salts include alkali metal salts, such as sodium salts, potassium salts, etc. and alkaline earth metal salts, such as calcium salts, magnesium salts, etc.

The compounds of the present invention may also be present in the free form, ester form (such as the ethyl ester), triglyceride form, diglyceride form, monoglyceride form, phospholipid form, or combinations thereof.

The compounds of the present invention can be also present as a reconstituted triglyceride, also known as transesterified or remodeled triglyceride. In the context of the present invention, the term “reconstituted triglyceride” (rTG) is to be understood as an oil comprising the triglyceride form, as the major component, and may also comprise the diglyceride form, the monoglyceride form, the ethyl ester form, or combinations thereof. The reconstituted triglyceride is obtained from the ethyl ester form through an enzymatic reaction, or a chemical transesterification, which is considered as a very common well-known process in the omega-3 industry field.

In the context of the present invention, when referring to the use of fatty acids according to the invention, the fatty acids may be in any of the forms defined in the preceding paragraphs.

In the context of the present invention, the term “Long COVID-19” is used as a synonym of “chronic COVID-19” and “post-COVID-19 syndrome”. “Long COVID-19” refers to a syndrome in patients having been diagnosed with acute COVID-19 based on clinical symptoms (with or without a positive SARS-COV-2 test, such as PCR, antigen, or antibody test), wherein one or more symptoms persist at least 12 weeks after the initial appearance of symptoms. This definition is based on the October 2020 guideline from the National Institute for Health and Care Excellence (NICE), Healthcare Improvement Scotland (SIGN), and the Royal College of General Practitioners (RCGP) in the UK.

### Treatment of Long COVID-19

In one aspect, the present invention concerns a composition for use in the treatment of Long COVID-19, wherein said composition comprises one or more of a specialized pro-resolving lipid mediator (SPM), an SPM precursor and/or a pharmaceutically acceptable salt thereof, as well as any stereoisomer thereof.

Depleted levels of SPMs are observed in Long COVID-19 patients along with elevated levels of pro-inflammatory cytokines. The active SPM precursors or the SPMs per se administered in accordance with the present invention may therefore alleviate this deficiency by re-establishing the balance between pro-inflammatory and pro-resolving substances. Administration of the SPM precursors is an interesting alternative to administration of the SPMs per se since the number of active precursors is limited compared to the number of SPMs per se. Furthermore, the resulting amounts of the individual SPMs are regulated by the physiological response to the active precursors.

Accordingly, in one embodiment, the composition of the invention comprises an SPM precursor and/or a pharmaceutically acceptable salt thereof, as well as any stereoisomer thereof. In a further embodiment, the composition comprises one or more compounds selected from the group consisting of 18-hydroxyeicosapentaenoic acid (18-HEPE), 17-hydroxydocosahexaenoic acid (17-HDHA), 14-hydroxydocosahexaenoic acid (14-HDHA) and pharmaceutically acceptable salts thereof, as well as any stereoisomers thereof. In another embodiment, the composition comprises two or more compounds selected from the group consisting of 18-hydroxyeicosapentaenoic acid (18-HEPE), 17-hydroxydocosahexaenoic acid (17-HDHA), 14-hydroxydocosahexaenoic acid (14-HDHA) and pharmaceutically acceptable salts thereof, as well as any stereoisomers thereof. In still another embodiment, the composition comprises three or more compounds selected from the group consisting of 18-hydroxyeicosapentaenoic acid (18-HEPE), 17-hydroxydocosahexaenoic acid (17-HDHA), 14-hydroxydocosahexaenoic acid (14-HDHA) and pharmaceutically acceptable salts thereof, as well as any stereoisomers thereof. In still a further embodiment, the composition further comprises EPA and/or DHA.

In one embodiment, the composition comprises 18-HEPE, 17-HDHA, and/or 14-HDHA in the ethyl ester form, triglyceride form, diglyceride form, monoglyceride form, phospholipid form, or combinations thereof.

In a further embodiment, the composition comprises 18-HEPE, 17-HDHA, and/or 14-HDHA in the ethyl ester form.

In yet a further embodiment, the composition is a reconstituted triglyceride comprising 18-HEPE, 17-HDHA, and/or 14-HDHA.

In another embodiment, the composition comprises 18-HEPE, 17-HDHA, and/or 14-HDHA in the phospholipid form.

In one embodiment, the composition comprises a purified fish oil, purified vegetable oil, and/or purified algae or microalgae oil comprising about 10% to 95% by weight of DHA, EPA or mixtures thereof, and about 0.0005% to about 1% by weight of specialized pro-resolving lipid mediator (SPM) precursor. In a further embodiment, the SPM precursors comprised in the purified fish oil, purified vegetable oil, and/or purified algae or microalgae oil are selected from the group consisting of 18-hydroxyeicosapentaenoic acid (18-HEPE), 17-hydroxydocosahexaenoic acid (17-HDHA), 14-hydroxydocosahexaenoic acid (14-HDHA) and pharmaceutically acceptable salts thereof, as well as any stereoisomers or mixtures thereof. In yet a further embodiment, the total dose of purified fish oil, purified vegetable oil, and/or purified algae or microalgae oil used in the treatment is in the range of 500 mg/day to 4000 mg/day, more preferably in the range of 1000 to 3500 mg/day. In another embodiment, the total dose of purified fish oil is 1500 or 3000 mg/day.

In some embodiments, the purified fish oil, purified vegetable oil, and/or purified algae or microalgae oil comprises DHA in an amount of about 5% to about 75%, such as about 10% to about 60%, by weight of the composition and/or EPA in an amount of about 5% to about 60%, such as about 10% to about 45%, by weight of the composition.

In some embodiments, the purified fish oil, purified vegetable oil, and/or purified algae or microalgae oil comprises 14-HDHA in an amount of about 0.001% to about 0.1% by weight of the composition, preferably from about 0.002% to about 0.08% by weight of the composition, more preferably from about 0.003%, to about 0.06% by weight of the composition, even more preferably from about 0.004% to about 0.02% by weight of the composition.

In some embodiments, the purified fish oil, purified vegetable oil, and/or purified algae or microalgae oil comprises 17-HDHA in an amount of about 0.001% to about 1.0% by weight of the composition, preferably from 0.002% to about 0.5% by weight of the composition, more preferably from about 0.005% to about 0.2% by weight of the composition, even more preferably from about 0.008% to about 0.04% by weight of the composition.

In some embodiments, the purified fish oil, purified vegetable oil, and/or purified algae or microalgae oil comprises 18-HEPE in an amount of about 0.001%, to about 1.0% by weight of the composition, preferably from 0.002% to about 0.5% by weight of the composition, more preferably from about 0.004% to about 0.2% by weight of the composition, even more preferably from about 0.005% to about 0.04% by weight of the composition.

In another embodiment, 18-HEPE, 17-HDHA, and 14-HDHA are obtained from fish oil. In a further embodiment, 18-HEPE, 17-HDHA, and 14-HDHA are obtained from algae or microalgae oil. In still a further embodiment, 18-HEPE, 17-HDHA, and 14-HDHA are obtained from vegetable oil.

In an alternative embodiment, the composition according to the invention comprises an SPM and/or a pharmaceutically acceptable salt thereof, as well as any stereoisomer thereof. In another embodiment, the composition comprises one or more compounds selected from the group consisting of 5S,6R, 15S-trihydroxy-7E,9E,11Z,13E-eicosatetraenoic acid (LXA4), 5S,14R,15S-trihydroxy-6E,8Z,10E, 12E-eicosatetraenoic acid (LXB4), 5S,6R, 15R-trihydroxy-7E,9E,11Z,13E-eicosatetraenoic acid (15-epi-LXA4), 5S, 14 R,15R-trihydroxy-6E,8Z,10E,12E-eicosatetraenoic acid (15-epi-LXB4), 5S, 12R, 18R-trihydroxy-6Z,8E,10E,14Z, 16E-eicosapentaenoic acid (RvE1), 5S, 12R, 18S-trihydroxy-6Z,8E,10E, 14Z, 16E-eicosapentaenoic acid (18S-RvE1), 5S, 18R-dihydroxy-6Z,8E,10E,14Z,16 E-eicosapentaenoic acid (RvE2), 17R,18R/S-dihydroxy-5Z,8Z,11Z,13E,15E-eicosapentaenoic acid (RvE3), 7S,8R,17S-trihydroxy-4Z,9E,11E,13Z,15E,19Z-docosahexaenoic acid (RvD1), 7S,16R,17 S-trihydroxy-4Z,8E,10Z,12E,14E,19Z-docosahexaenoic acid (RvD2), 4S,11R, 17S-trihydroxy-5Z,7E,9E,13Z,15E, 19Z-docosahexaenoic acid (RvD3), 4S,5R,17S-trihydroxy-6E,8E,10Z,13Z,15E,19Z-docosahexaenoic acid (RvD4), 7S, 17S-dihydroxy-4Z,8E,10Z,13Z,15E,19Z-docosahexaenoic acid (RvD5), 4S,17S-dihydroxy-4Z,8E,10Z,13Z,15E,19Z-docosahexaenoic acid (RvD6), 7S,8R,17R-trihydroxy-4Z,9E,11 E,13Z,15E, 19Z-docosahexaenoic acid (17R-RvD1), 7S, 16R, 17 R-trihydroxy-4Z,8E,10Z,12E,14E, 19Z-docosahexaenoic acid (17R-RvD2), 4S,11R,17 R-trihydroxy-5Z,7E,9E,13Z,15E,19Z-docosahexaenoic acid (17R-RvD3), 4S,5R, 17 R-trihydroxy-6E,8E,10Z,13Z,15E,19Z-docosahexaenoic acid (17R-RvD4), 7S,17R-dihydroxy-4Z,8E,10Z,13Z,15E,19Z-docosahexaenoic acid (17R-RvD5), 4S,17R-dihydroxy-4Z,8E,10Z,13Z,15E,19Z-docosahexaenoic acid (17R-RvD6), 7,13R,20-trihydroxy-8E,10Z,14E,16Z,18E-docosapentaenoic acid (RvT1), 7,8, 13R-trihydroxy-9E,11E,14E, 16Z,19Z-docosapentaenoic acid (RvT2), 7,12,13R-trihydroxy-8Z,10E,14E,16Z,19Z-docosapentaenoic acid (RvT3), 7,13R-dihydroxy-8E,10Z,14E,16Z,19Z-docosapentaenoic acid (RvT4), 7,8,17-trihydroxy-8,10, 13, 15, 19-docosapentaenoic acid (RvD1n-3 DPA), 7,16,17-trihydroxy-8,10, 12, 14, 19-docosapentaenoic acid (RvD2n-3 DPA), 7,17-dihydroxy-8, 10, 13, 15, 19-docosapentaenoic acid (RvD5n-3 DPA), 10R, 17S-dihydroxy-4Z,7Z,11E,13E,15Z,19Z-docosahexaenoic acid (PD1), 10S, 17S-dihydroxy-4Z,7Z,11E,13E, 15Z,19Z-docosahexaenoic acid (PDX), 10R,17S,22-trihydroxy-4Z,7Z,11E,13E,15Z,19Z-docosahexaenoic acid (22-hydroxy-PD1), 10R, 17 R-dihydroxy-4Z,7Z,11E,13E, 15Z,19Z-docosahexaenoic acid (17-epi-PD1), 10S,17S-dihydroxy-4Z,7Z,11E,13E,15Z,19Z-docosahexaenoic acid (10-epi-PD1), 10, 17-dihydroxy-7,11, 13, 15, 19-docosapentaenoic acid (PD1n-3 DPA), 16, 17-dihydroxy-7,10, 12,14, 19-docosapentaenoic acid (PD2n-3 DPA), 7R,14S-dihydroxy-4Z,8E,10E,12Z,16Z,19Z-docosahexaenoic acid (MaR1), 13R,14S-dihydroxy-4Z,7Z,9E,11 E,16Z,19Z-docosahexaenoic acid (MaR2), 7S,14S-dihydroxy-4Z,8E,10E,12Z,16Z,19Z-docosahexaenoic acid (7-epi-MaR1), 14S,22-dihydroxy-4Z,7Z,10Z,12E,16Z,19Z-docosahexaenoic acid (MaR-L1), 14R,22-dihydroxy-4Z,7Z,10Z,12E,16Z,19Z-docosahexaenoic acid (MaR-L2), and 7S,14S-dihydroxy-8E,10E,12Z,16Z,19Z-docosapentaenoic acid (MaR1n-3 DPA).

In yet a further embodiment, the composition comprises one or more compounds selected from the group consisting of 5S,6R, 15S-trihydroxy-7E,9E,11Z,13E-eicosatetraenoic acid (LXA4), 5S,14R, 15S-trihydroxy-6E,8Z,10E,12E-eicosatetraenoic acid (LXB4), 5S,6R,15R-trihydroxy-7E,9E,11Z,13E-eicosatetraenoic acid (15-epi-LXA4), 5S,14R, 15R-trihydroxy-6E,8Z,10E,12E-eicosatetraenoic acid (15-epi-LXB4), 5S, 12R,18R-trihydroxy-6Z,8E,10E,14Z,16E-eicosapentaenoic acid (RvE1), 5S,12R, 18S-trihydroxy-6Z,8E,10E,14Z,16E-eicosapentaenoic acid (18S-RvE1), 5S, 18R-dihydroxy-6Z,8E, 10E,14Z,16E-eicosapentaenoic acid (RvE2), 17R, 18R/S-dihydroxy-5Z,8Z,11Z,13E,15E-eicosapentaenoic acid (RvE3), 7S,8R,17S-trihydroxy-4Z,9E,11E,13Z,15E,19Z-docosahexaenoic acid (RvD1), 7S,16R, 17S-trihydroxy-4Z,8E,10Z,12E,14E,19Z-docosahexaenoic acid (RvD2), 4S, 11R, 17S-trihydroxy-5Z,7E,9E,13Z,15E,19Z-docosahexaenoic acid (RvD3), 4S,5R,17S-trihydroxy-6E,8E,10Z,13Z,15E,19Z-docosahexaenoic acid (RvD4), 7S, 17 S-dihydroxy-4Z,8E,10Z,13Z,15E,19Z-docosahexaenoic acid (RvD5), 4S,17S-dihydroxy-4Z,8E,10Z,13Z,15E,19Z-docosahexaenoic acid (RvD6), 7S,8R,17R-trihydroxy-4Z,9E,11 E,13Z,15E,19Z-docosahexaenoic acid (17R-RvD1), 7S, 16R,17 R-trihydroxy-4Z,8E,10Z,12E,14E, 19Z-docosahexaenoic acid (17R-RvD2), 4S,11R, 17 R-trihydroxy-5Z,7E,9E,13Z,15E,19Z-docosahexaenoic acid (17R-RvD3), 4S,5R,17 R-trihydroxy-6E,8E,10Z,13Z,15E,19Z-docosahexaenoic acid (17R-RvD4), 7S, 17R-dihydroxy-4Z,8E,10Z,13Z,15E,19Z-docosahexaenoic acid (17 R-RvD5), 4S, 17R-dihydroxy-4Z,8E,10Z,13Z,15E,19Z-docosahexaenoic acid (17R-RvD6), 7,13R,20-trihydroxy-8E,10Z,14E,16Z,18E-docosapentaenoic acid (RvT1), 7,8,13R-trihydroxy-9E,11E,14E,16Z,19Z-docosapentaenoic acid (RvT2), 7,12,13R-trihydroxy-8Z,10E,14E,16Z,19Z-docosapentaenoic acid (RvT3), 7,13R-dihydroxy-8E,10Z,14E,16Z,19Z-docosapentaenoic acid (RvT4), 7,8,17-trihydroxy-8,10, 13, 15, 19-docosapentaenoic acid (RvD1n-3 DPA), 7,16,17-trihydroxy-8, 10, 12, 14, 19-docosapentaenoic acid (RvD2n-3 DPA), 7,17-dihydroxy-8,10, 13, 15, 19-docosapentaenoic acid (RvD5n-3 DPA), 10R,17S-dihydroxy-4Z,7Z,11 E,13E,15Z,19 Z-docosahexaenoic acid (PD1), 10S, 17S-dihydroxy-4Z,7Z,11E,13E, 15Z,19Z-docosahexaenoic acid (PDX), 10R,17S,22-trihydroxy-4Z,7Z,11E,13E,15Z,19Z-docosahexaenoic acid (22-hydroxy-PD1), 10R,17R-dihydroxy-4Z,7Z,11E,13E,15Z,19Z-docosahexaenoic acid (17-epi-PD1), 10S,17S-dihydroxy-4Z,7Z,11E,13E, 15Z,19Z-docosahexaenoic acid (10-epi-PD1), 10, 17-dihydroxy-7,11, 13, 15, 19-docosapentaenoic acid (PD1n-3 DPA), 16, 17-dihydroxy-7,10, 12,14, 19-docosapentaenoic acid (PD2n-3 DPA), 14S,22-dihydroxy-4Z,7Z,10Z,12E, 16Z,19Z-docosahexaenoic acid (MaR-L1), and 14R,22-dihydroxy-4Z,7Z,10Z,12E,16Z,19Z-docosahexaenoic acid (MaR-L2).

In a more preferred embodiment, the composition comprises 18-HEPE, 17-HDHA, 14-HDHA, and one or more compounds selected from the group consisting of 5S,6R, 15 S-trihydroxy-7E,9E,11Z,13E-eicosatetraenoic acid (LXA4), 5S, 14R,15S-trihydroxy-6E,8Z,10E, 12E-eicosatetraenoic acid (LXB4), 5S,6R, 15R-trihydroxy-7E,9E,11Z,13E-eicosatetraenoic acid (15-epi-LXA4), 5S, 14R,15R-trihydroxy-6E,8Z,10E,12E-eicosatetraenoic acid (15-epi-LXB4), 5S,12R, 18 R-trihydroxy-6Z,8E, 10E,14Z,16E-eicosapentaenoic acid (RvE1), 5S,12R, 18S-trihydroxy-6Z,8E,10E,14Z, 16 E-eicosapentaenoic acid (18S-RvE1), 5S, 18R-dihydroxy-6Z,8E,10E,14Z, 16E-eicosapentaenoic acid (RvE2), 17R,18R/S-dihydroxy-5Z,8Z,11Z,13E, 15E-eicosapentaenoic acid (RvE3), 7S,8R,17 S-trihydroxy-4Z,9E,11 E,13Z,15E,19Z-docosahexaenoic acid (RvD1), 7S, 16R, 17S-trihydroxy-4Z,8E,10Z,12E,14E,19Z-docosahexaenoic acid (RvD2), 4S, 11R,17S-trihydroxy-5Z,7E,9E,13Z,15E,19Z-docosahexaenoic acid (RvD3), 4S,5R,17S-trihydroxy-6E,8E,10Z,13Z,15E,19Z-docosahexaenoic acid (RvD4), 7S, 17 S-dihydroxy-4Z,8E,10Z,13Z,15E,19Z-docosahexaenoic acid (RvD5), 4S,17 S-dihydroxy-4Z,8E,10Z,13Z,15E,19Z-docosahexaenoic acid (RvD6), 7S,8R,17R-trihydroxy-4Z,9E,11 E,13Z,15E, 19Z-docosahexaenoic acid (17R-RvD1), 7S, 16R, 17 R-trihydroxy-4Z,8E,10Z,12E,14E, 19Z-docosahexaenoic acid (17R-RvD2), 4S,11R,17 R-trihydroxy-5Z,7E,9E,13Z,15E,19Z-docosahexaenoic acid (17R-RvD3), 4S,5R,17 R-trihydroxy-6E,8E,10Z,13Z,15E,19Z-docosahexaenoic acid (17R-RvD4), 7S,17R-dihydroxy-4Z,8E,10Z,13Z,15E,19Z-docosahexaenoic acid (17R-RvD5), 4S,17R-dihydroxy-4Z,8E,10Z,13Z,15E,19Z-docosahexaenoic acid (17R-RvD6), 7,13R,20-trihydroxy-8E,10Z,14E,16Z,18E-docosapentaenoic acid (RvT1), 7,8, 13R-trihydroxy-9E,11E,14E,16Z,19Z-docosapentaenoic acid (RvT2), 7,12,13R-trihydroxy-8Z,10E,14E,16Z,19Z-docosapentaenoic acid (RvT3), 7,13R-dihydroxy-8E,10Z,14E,16Z,19Z-docosapentaenoic acid (RvT4), 7,8,17-trihydroxy-8,10, 13, 15, 19-docosapentaenoic acid (RvD1n-3 DPA), 7,16,17-trihydroxy-8,10, 12, 14, 19-docosapentaenoic acid (RvD2n-3 DPA), 7,17-dihydroxy-8, 10, 13, 15, 19-docosapentaenoic acid (RvD5n-3 DPA), 10R,17S-dihydroxy-4Z,7Z,11E,13E,15Z,19Z-docosahexaenoic acid (PD1), 10S, 17S-dihydroxy-4Z,7Z,11E,13E, 15Z,19Z-docosahexaenoic acid (PDX), 10R,17S,22-trihydroxy-4Z,7Z,11E,13E,15Z,19Z-docosahexaenoic acid (22-hydroxy-PD1), 10R,17R-dihydroxy-4Z,7Z,11E,13E,15Z,19Z-docosahexaenoic acid (17-epi-PD1), 10S, 17S-dihydroxy-4Z,7Z,11E,13E,15Z,19Z-docosahexaenoic acid (10-epi-PD1), 10, 17-dihydroxy-7,11,13,15, 19-docosapentaenoic acid (PD1n-3 DPA), 16, 17-dihydroxy-7,10, 12,14, 19-docosapentaenoic acid (PD2n-3 DPA), 14S,22-dihydroxy-4Z,7Z,10Z,12E, 16Z,19Z-docosahexaenoic acid (MaR-L1), and 14R,22-dihydroxy-4Z,7Z,10Z,12E,16Z, 19Z-docosahexaenoic acid (MaR-L2).

Preparation of SPMs and their active precursors is known in the art, including WO 2013/170006. SPMs and their active precursors may also be administered in a form, such as an ester of the fatty acid, that upon entry into the body is hydrolysed to the fatty acid.

Patients suffering from Long COVID-19 may suffer from one or more symptoms, such as fatigue, dyspnea, asthenia, joint pain, chest pain, insomnia, headaches, and attention disorders. In the context of the present invention, when referring to “treatment of Long COVID-19”, alleviation of at least one symptom is achieved. In one embodiment, at least one symptom disappears completely. In a further embodiment, at least two symptoms are alleviated. In still a further embodiment, at least two symptoms disappear completely. In yet a further embodiment, at least two symptoms, such as fatigue and dyspnea, are alleviated, with at least one of them disappearing completely. In another embodiment, at least three symptoms are alleviated. In still another embodiment, at least three symptoms disappear completely. In yet another embodiment, at least three symptoms are alleviated, with at least one or two symptoms disappearing completely. In still a further embodiment, all symptoms are alleviated. In yet a further embodiment, all symptoms disappear completely.

The dose of SPM or SPM precursor used in the treatment may be varied according to the development of the condition. In one embodiment, the total dose of specialized pro-resolving lipid mediator (SPM), SPM precursor and/or pharmaceutically acceptable salt thereof, as well as any stereoisomer thereof, is in the range of 0.01 mg to 10 mg/day. In a further embodiment, the total dose is in the range of 0.02 to 5 mg/day. In still a further embodiment, the total dose is in the range of 0.03 to 3 mg/day.

The daily dosage of SPM or SPM precursor may be administered once per day or be divided into several administrations per day. In one embodiment, the total daily dosage is administered in divided doses 1 to 4 times a day. In a further embodiment, the total daily dosage is administered in divided doses 2 to 3 times a day. In another embodiment, the total daily dosage is administered in divided doses 3 times a day.

### Treatment of Inflammation Associated with Long COVID-19

Long COVID-19 is associated with inflammation and SPMs are known to play a role in the active resolution of inflammation. Accordingly, in one aspect, the invention concerns a composition for use in the treatment of inflammation associated with Long COVID-19, wherein said composition comprises a specialized pro-resolving lipid mediator (SPM), an SPM precursor and/or a pharmaceutically acceptable salt thereof, as well as any stereoisomer thereof.

### Combination with a Non-Steroidal Anti-Inflammatory Drug (NSAID).

Acetylsalicylic acid and other non-steroidal anti-inflammatory drugs function as co-enzymes for lipoxygenase and/or cyclooxygenase and thus contribute to the bio-synthesis of SPMs in the cascade having EPA and/or DHA as the substrate. Suitable non-steroidal anti-inflammatory drugs include ibuprofen, flurbiprofen, naproxen, indomethacin, diclofenac, celecoxib, and acetylsalicylic acid. Accordingly, in one embodiment the composition for use in treating Long COVID-19 according to the invention is further combined with a non-steroidal anti-inflammatory drug. The non-steroidal anti-inflammatory drug may be comprised in the same composition as the SPMs and/or SPM precursors or may be administered separately. In a further embodiment, said non-steroidal anti-inflammatory drug is selected from ibuprofen, flurbiprofen, naproxen, indomethacin, diclofenac, celecoxib, and acetylsalicylic acid. In still a further embodiment, said non-steroidal anti-inflammatory drug is acetylsalicylic acid. In another embodiment, the acetylsalicylic acid is administered at a dose of 50 to 150 mg. In yet another embodiment, the acetylsalicylic acid is administered at a dose of 75 to 125 mg. In still a further embodiment, the acetylsalicylic acid is administered at a dose of approximately 100 mg.

### Formulation

The compounds comprised in the compositions of the present invention are intended for use as a medicament or as a food supplement. The compounds of the invention may in principle be applied on their own, but they are preferably formulated with a pharmaceutically acceptable carrier or a carrier acceptable for food supplements. An acceptable carrier is an inert carrier suitable for each administration method and can be formulated into conventional preparations (tablets, granules, capsules, powder, solution, suspension, emulsion, injection, infusion, etc.). One particular example of a suitable formulation is a soft capsule containing the SPMs and/or active precursors as a purified fish oil, purified vegetable oil, and/or purified algae or microalgae oil. As an inert carrier there may be mentioned, for example, a filler, a binder, an excipient, a lubricant, a disintegrant and the like, which are acceptable. When they are used as an injection solution or an infusion solution, they can be formulated by using distilled water for injection, physiological saline, or an aqueous glucose solution.

In one embodiment, the composition of the invention comprises an antioxidant. Without being bound by a particular theory, the antioxidant has been found to improve stability of the composition.

### Administration

The administration method of the compositions of the present invention is not particularly limited, and a usual oral or parenteral administration method (intravenous, intramuscular, subcutaneous, percutaneous, intranasal, transmucosal, enteral, etc.) can be applied.

### EXAMPLES

### Example 1—Study of the Effects of a Food Supplement on Inflammatory and Pro-Resolution Factors in Patients With Long COVID-19

### Objective

Evaluation of the effect of a food supplement rich in omega-3 fatty acids on different pro-inflammatory lipid mediators and pro-resolution mediators in patients diagnosed with Long COVID-19. The effect of the food supplement on fatigue and dyspnea in patients with Long COVID-19, as well its safety and tolerability, will also be evaluated.

Study Design

Pilot, multicenter, randomized, double-blind, parallel design, placebo-controlled clinical trial.

### Study population

37 patients with Long COVID-19 are examined (randomization: 3:3:1 [16/16/5]). Additional exploratory cohort of 16 patients.

### Treatment Groups

| | | No capsules/8 h | |
|---|---|---|---|
| Group | Product | Supplement | Placebo |
| A | “Omega-3 food supplement” 1000 mg | 2 | 0 |
| | every 8 hours | | |
| B | “Omega-3 food supplement” 500 mg | 1 | 1 |
| | every 8 hours | | |
| C | Placebo every 8 hours | 0 | 2 |

| | | |
|---|---|---|
| X | “Omega-3 food supplement” 500 mg | 1 |
| | every 24 hours | |

The treatment allocation was made by randomly assigning each subject any of the treatments or the placebo group. The randomization ratio was 3:3:1:3 [16/16/5/16]) 37 patients were included in groups A, B y C (randomization 3:3:1)
Group A N=16 patients
Group B N=16 patients.
Group C Placebo N=5 patients

This study was double-blind and placebo-controlled.

Each capsule of 500 mg Omega-3 food supplement contains the following components:
Omega-3 fatty acids: 337.5 mg
   EPA: 100 mg
   DHA: 162.5 mg
SPM precursors:
   17-HDHA 120 μg
   18-HEPE 112.5 μg
   14-HDHA 55 μg

This composition will be referred to as the “investigational product” or “IP” in the following.

### Inclusion Criteria

To participate in the study, patients must meet all of the following:
1) Patients of both sexes with Long COVID-19 between 18 and 70 years old.
   a) Patients with clinical criteria of having previously suffered from acute Covid 19 (with confirmed diagnosis using diagnostic techniques and systems for COVID-19 [PCR technique, rapid antigen detection tests, serological antibody detection test]) and who remain with symptoms more than 12 weeks after the onset of symptoms
   b) Presence of fatigue/asthenia, dyspnea and one of the following symptoms:
      Headaches
      General discomfort.
      Having a feeling of low spirits
      Muscle pains
2) Body mass index between 18.5 and 30 kg/m²
3) Ability to provide informed consent
4) Women participating in the study must meet one of these two conditions:
   Women without the possibility of becoming pregnant, defined as any woman who has undergone surgical sterilization or who has been postmenopausal for more than two years.
   Women with the possibility of becoming pregnant, as long as they have a negative pregnancy test at the beginning of the study (screening and qualification period) and regularly use a highly effective contraceptive method (i.e. hormonal contraception, intrauterine device, condoms in combination with spermicidal cream, sterilization of the male partner [vasectomy] or total sexual abstinence) while they are participating in the study. The use of highly effective contraceptives should continue for at least 3 months after the last dose of study treatment.

### Exclusion Criteria

To participate in the study, patients should not meet any of the following:
1) Pregnant or lactating women
2) Impossibility of guaranteeing an effective contraceptive method
3) Subjects enrolled in a clinical trial
4) Subjects who have completed another clinical trial in the last 4 weeks prior to inclusion
5) Any disease or condition that may significantly compromise the hematological, renal, endocrine, pulmonary, hepatic, gastrointestinal, cardiovascular, immune, central nervous, dermatological or any other body system, with the exception of the conditions defined in the inclusion criteria
6) The use of immunosuppressive drugs
7) Hypersensitivity, allergy or idiosyncratic reaction to omega 3 acids; allergy to fish oil or soybean oil

### Evaluation Criteria

### Efficacy Variables

### Primary Endpoint:

Change from baseline until twelve weeks (day 84) after treatment of the following pro-inflammatory lipid mediators and pro-resolution mediators of the inflammatory response:
   Fatty acids: EPA, DHA, ARA, DPA (docosapentaenoic acid)
   SPM precursors: 17-HDHA, 18-HEPE, 14-HDHA
   Resolvins: RvE1, RvD1, RvD2, RvD3, RvD4, RvD5
   Maresins: MaR1, MaR2
   Protectins: PD1, PDX
   Lipoxins: LXA4, LXB4
   Prostaglandins: PGE2, PGD2, PGF2a
   Tromboxanes: TXB2
   Leukotrienes: LTB4

### Secondary Endpoints:

Change from baseline until 4 and 12 weeks on the Fatigue Severity Scale (FSS): The FSS test measures fatigue in a unidimensional scale. It consists in 9 questions with 7 possible answers each which quantifies each item in a 1 to 7 scale. The evolution of the mean scores from baseline to visit 2 (4th week of treatment, day 28) and to the end of the study (day 84 of treatment) is calculated.
Change from baseline until 4 and 12 weeks on the Modified Dyspnea scale (mMRC): The scale includes 5 degrees of physical activity that could cause dyspnea. The scale punctuates the dyspnea in a range from 0 (No exercise cause dyspnea) to 4 (the dyspnea prevents the patients going out of the house or performing routine daily activities like dressing up. The baseline results are compared to the scores at visit 2 (day 28) and at the end of the study (day 84).
Change from baseline until 4 weeks (day 28) after treatment of the pro-inflammatory lipid mediators and pro-resolution mediators of the inflammatory response mentioned above

### Safety and Tolerability Variables

Incidence of serious adverse events
Incidence of adverse events
Clinical evaluation, arterial pressure and heart rate

### RESULTS

Regarding the primary endpoint of the study, the evolution of pro-inflammatory and pro-resolution/anti-inflammatory mediators after 12 weeks of treatment, there were no significant differences in the majority of the metabolites studied. Group A is the only where all pro-inflammatory means are reduced. At 12 weeks, all groups, even the placebo (C) group, experience a reduction in the pro-inflammatory and an increase in the pro-resolving/anti-inflammatory mediators.

Overall, all PUFAs and monohydroxylated mediators 14-HDHA, 17-HDHA and 18-HEPE, tend to increase during the study in all groups. The highest dose group experienced the highest, statistically significant increase of 18-HEPE at 12 weeks compared with the rest of the groups.

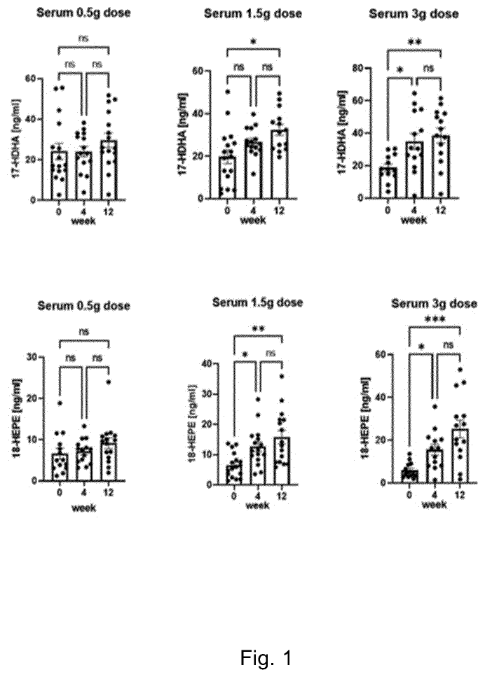

The intragroup differences are statistically significant for 17-HDHA between baseline and the second and third visit in the highest dose group and between baseline and the last visit in the medium dose group (FIG. 1). Likewise, 18-HEPE increases significantly between baseline and the 2nd and last visit in the two highest dose group but not in the lowest dose group (FIG. 1). There were no significant differences after the IP consumption for 14-HDHA in any of the groups.

Concerning the composite variables at 4 weeks, anti-inflammatory mediators rise in groups A, B and X and decrease in the placebo group. At week 12, anti-inflammatory variables rise and the pro-inflammatory decrease in all groups. When comparing the evolution of the composite variables between the treatment groups at the end of the study, the pro-inflammatory mediators were significantly lower in the 3 g/day group compared with groups C (Placebo) (p=0.036) and B (p=0.04), but not compared with group X.

### Ratio Monohydroxylated SPM Precursors Vs Pro-Inflammatory Mediators in Serum

To compare the evolution of the inflammatory status of the patients and their capability of resolution, the ratio of SMP precursors and pro-inflammatory mediators was compared for the 3active ingredient groups over the course of the study. The total concentration of each pro-resolution monohydroxylated SPM, 14-HDHA, 18-HEPE and 17-HDHA was added and divided by the total number of pro-inflammatory metabolites, PGE2, PGD2, PGF2α, TXB2.

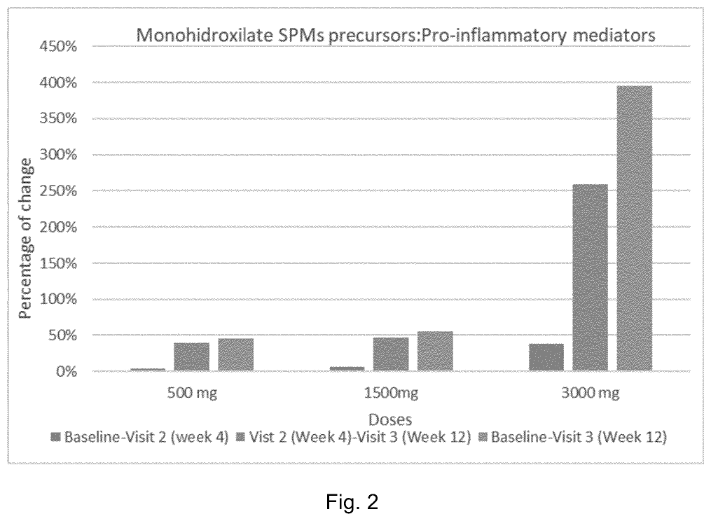

The total serum SPM precursors: pro-inflammatory ratio increases during the study in a dose dependent manner for all doses used (FIG. 2). The more significant increment of the ratio occurs in the late stages of the study, between visits 2 (after 4 weeks of treatment) and 3 (12 weeks after the beginning of the treatment). The ratio pro-resolution: pro-inflammatory increases, during the last period, a 259% for the highest dose group (A), 47% for the medium dose group (B), and 40% for the lowest dose group (X).

Table 2 depicts the results.

| | Group X (500 mg) | | | Group B (1500 mg) | | | Group A (3000 mg) | | |
|---|---|---|---|---|---|---|---|---|---|
| Serum | Baseline | Visit | % | Baseline | Visit | % | Baseline | Visit | % |

Baseline-Visit 2 (week 4)

| | | | | | | | | | |
|---|---|---|---|---|---|---|---|---|---|
| Monohydroxylated | 167.3 | 201.9 | 21% | 169.9 | 225.4 | 33% | 184.4 | 250.1 | 36% |
| Monohydroxylated/pro- | 2.20 | 2.28 | 4% | 2.65 | 2.82 | 7% | 1.14 | 1.57 | 38% |
| inflammatory | | | | | | | | | |

Visit 2 (week 4) - Visit 3 (week 12)

| | | | | | | | | | |
|---|---|---|---|---|---|---|---|---|---|
| Monohydroxylated | 201.9 | 214.8 | 6% | 225.4 | 278.1 | 23% | 250.1 | 290.9 | 16% |
| Monohydroxylated/pro- | 2.28 | 3.19 | 40% | 2.82 | 4.14 | 47% | 1.57 | 5.62 | 259% |
| inflammatory | | | | | | | | | |

Baseline - Visit 3 (week 12)

| | | | | | | | | | |
|---|---|---|---|---|---|---|---|---|---|
| Monohydroxylated | 167.3 | 214.8 | 28% | 169.9 | 278.1 | 64% | 184.4 | 290.9 | 58% |
| Pro- | 2.20 | 3.19 | 45% | 2.65 | 4.14 | 56% | 1.14 | 5.62 | 395% |
| inflammatory/monohydroxylated | | | | | | | | | |

### Composite Metabolomes Vs Prostaglandins

Since the prostaglandins are some of the most relevant determinants for the evolution of the inflammatory response, the ratio of their concentrations in serum and plasma was compared with the 14-HDHA, 17-HDHA and 18-HEPE metabolomes during the study.

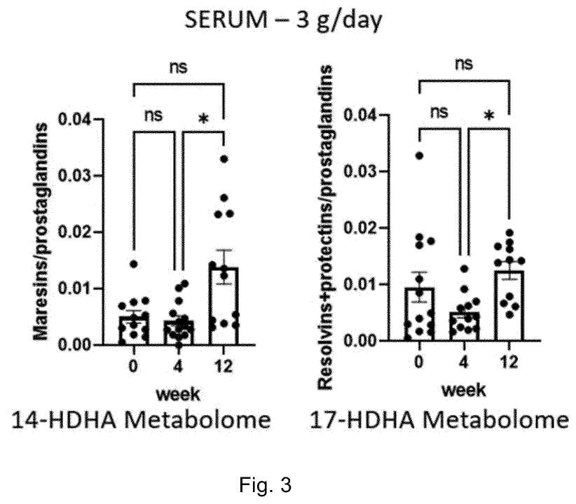

The 14-HDHA metabolome comprises Maresins 1 and 2, the 17-HDHA metabolome comprises Resolvins, RvD1, RvD2, RvD3, RvD4, RvD5 and protectins PD1 and PDx, whereas the 18-HEPE metabolome comprises RvE1. There were no significant differences between baseline and visit 2, where a slight decrease in the ratio is observed, or baseline and visit 3 in any of the ratios. However, statistically significant differences were found between weeks 4 and 12 (FIG. 3), which points to a delayed action of the IP, which exerts its action after several weeks of treatment. During the first weeks of the study, no significant differences were observed regarding the 18-HEPE metabolome.

### Secondary Objectives

To determine the effect of the IP on the clinical manifestation of long COVID-19, as secondary objectives of the study, its effects on the patients' fatigue and dyspnea, two of the most prevalent symptoms observed in these patients, was assessed. The secondary efficacy variables are:
Changes in the Fatigue Severity Scale (FSS) scores from baseline until weeks 4 and 12.
Changes form baseline until weeks 4 and 12 in the mMRC (Modified Medical Research Council) Dyspnoea Scale.

Both scales are commonly used and validated methods to assess either fatigue or the degree of functional disability due to dyspnea.

The evolution of these clinical variables was analysed for the 4 groups of treatment using a mixed general linear model. Differences between the baseline FSS scores and 4 and 12 weeks after treatment were calculated. All groups show a tendency to improve the fatigue symptoms included in the FSS questionnaire, but no significant differences are detected among the 4 treatment groups.

Differences between baseline and week 4 and 12 in the mMRC scale scores were calculated for each individual patient. The Chi-square was used for the analysis of the differences between treatments. For the differences between baseline and week 12, X-squared=8.2496 and p-value=0.509, and between baseline and week 4 X-squared=7.3615 and p-value=0.600. A slight improvement can be observed for each group, in terms of frequency and percentage of patients in each grade of the scale, but there were no statistically significant differences in the evolution of the mMRC scores among the 4 treatment groups during the study. The trend, however, was towards an improvement in the clinical parameters.

### Conclusions

The total serum SPM precursors: pro-inflamatory mediators ratio increases during the study in a dose dependent manner for all doses used. The more significant increment of the ratio occurs in the later stages of the study, between visits 2 (after 4 weeks of treatment) and 3 (12 weeks after the beginning of the treatment). The ratio pro-resolution: pro-inflammatory increases, during the last period, a 259% for the highest dose group (A), 47% for the medium dose group (B), and 40% for the lowest dose group (X).

These results reaffirm the efficacy of the IP in the improvement of the metalipidinomic parameters.

A trend towards an improvement in the clinical parameters was also observed.

### REFERENCES

[1] Flower RJ. Prostaglandins, bioassay, and inflammation. Br J Pharmacol 2006; 147:82-192. https://doi.org/10.1038/sj.bjp.0706506.

[2] Samuelsson B. Roleofbasicscienceinthedevelopmentofnewmedicines:examples from the eicosanoid field. J Biol Chem 2012;287 (13): 10070-80.

[3] Dinarello CA, Simon A, van der Meer JWM. Treating inflammation by blocking interleukin-1 in a broad spectrum of diseases. Nat Rev Drug Discov 2012;11 (8): 633-52.

[4] Serhan CN, Savill J. Resolution of inflammation: the beginning programs the end. Nat Immunol 2005;6 (12): 1191-7.

[5] Maderna P, Godson C. Lipoxins: resolutionary road. Br J Pharmacol 2009;158:947-59. https://doi.org/10.1111/j.1476-5381.2009.00386.

[6] Tabas I, Glass CK. Anti-inflammatory therapy in chronic disease: challenges and opportunities. Science 2013;339 (6116): 166-72. https://doi.org/10.1126/science. 1230720.

[7] Serhan CN, Clish CB, Brannon J, Colgan SP, Chiang N, Gronert K. Novel functional sets of lipid-derived mediators with antiinflammatory actions generated from omega-3 fatty acids via cyclooxygenase 2-nonsteroidal antiinflammatory drugs and transcellular processing. J Exp Med 2000;192:1197-204. https://doi.org/10.1084/jem.192.8.1197.

[8] SerhanCN, HongS,GronertK,ColganSP, DevchandPR, MirickG,MoussignacR-L. Resolvins: a family of bioactive products of omega-3 fatty acid transformation cir-cuits initiated by aspirin treatment that counter pro-inflammation signals. J Exp Med 2002;196:1025-37. https://doi.org/10.1084/jem.20020760.

[9] Lands WE M. Fish, Omega-3 and Human Health 2nd edn (AOCS Press, 2005) p 34-45.

[10] SerhanCN, ChiangN.Resolutionphaselipidmediatorsofinflammation: agonistsof resolution. Curr Opin Pharmacol 2013;13 (4): 632-40.

[11] Serhan CN, Levy BD. Resolvins in inflammation: emergence of the pro-resolving superfamily of mediators. J Clin Invest 2018;128 (7): 2657-69. https://doi.org/10. 1172/JCI97943.

[12] SerhanCN.Pro-resolvinglipidmediatorsareleadsforresolutionphysiology.Nature 2014;510 (7503): 92-101. https://doi.org/10.1038/nature13479.

[13] SerhanCN. Treatinginflammationandinfectioninthe21stcentury: newhintsfrom decoding resolution mediators and mechanisms. FASEB J 2017;31 (4): 1273-88.

[14] HuangC, WangY,Lix, RenL,ZhaoJ, HuYi,ZhangLi,FanG,XuJ,GuX, ChengZ, Yu T, Xia J, Wei Y, Wu W, Xie X, Yin W, Li H, Liu M, Xiao Y, Gao H, Guo Li, Xie J, Wang G, Jiang R, Gao Z, Jin Qi, Wang J, Cao B. Clinical features of patients infected with 2019 novel coronavirus in Wuhan, China. Lancet 2020;395 (10223): 497-506. https://doi.org/10.1016/S0140-6736 (20) 30183-5.

[15] ZhouF, YuT, DuR, FanG, LiuY, LiuZ, XiangJ, WangY, SongB, Gux, GuanL, Wei Y, Li H, Wu X, Xu J, Tu S, Zhang Yi, Chen H, Cao B. Clinical course and risk factors for mortality of adult inpatients with COVID-19 in Wuhan, China: a retrospective cohort study. Lancet 2020;395 (10229): 1054-62. https://doi.org/10.1016/S0140-6736 (20) 30566-3.

[16] Nicolai Leo, Leunig Alexander, Brambs Sophia, Kaiser Rainer, Weinberger Tobias, Weigand Michael, Muenchhoff Maximilian, Hellmuth Johannes C, Ledderose Stephan, Schulz Heiko, Scherer Clemens, Rudelius Martina, Zoller Michael, Höchter Dominik, Keppler Oliver, Teupser Daniel, Zwißler Bernhard, von Bergwelt-Baildon Michael, Kääb Stefan, Massberg Steffen, Pekayvaz Kami, Stark Konstantin. Immunothrombotic Dysregulation in COVID-19 Pneumonia Is Associated With Respiratory Failure and Coagulopathy. Circulation 2020;142 (12): 1176-89.

[17] Cherpokova D, Jouvene CC, Libreros S, DeRoo EP, Chu L, de la Rosa X, Norris PC, Wagner DD, Serhan CN. Resolvin D4 attenuates the severity of pathological thrombosis in mice. Blood 2019 | VOLUME 00, DOI: 10.1182/blood.2018886317.

[18] Elajami TK, Colas RA, Dalli J, Chiang N, Serhan CN, Welty FK. Specialized pro-resolving lipid mediators in patients with coronary artery disease and their po-tential for clot remodelling. FASEB J 2016;30:2792-801. https://doi.org/10.1096/fj.201500155R.

1. A composition for use in the treatment of Long COVID-19, wherein said composition comprises one or more of a specialized pro-resolving lipid mediator (SPM), an SPM precursor and/or a pharmaceutically acceptable salt thereof, as well as any stereoisomer thereof, wherein Long COVID-19 patients are patients having been diagnosed with acute COVID-19 based on clinical symptoms, wherein one or more symptoms persist at least 12 weeks after the initial appearance of symptoms.

2-16. (canceled)
